(19) United States
(12) Patent Application Publication
(10) Pub. No.: US 2011/0045444 A1
(43) Pub. Date: Feb. 24, 2011

(54) DENTAL FILLING COMPOSITION COMPRISING HYPERBRANCHED COMPOUND

(76) Inventors: Prabhakara S. Rao, Maplewood, MN (US); Steven M. Aasen, Woodbury, MN (US); Belma Erdogan-Haug, St. Paul, MN (US); Babu N. Gaddam, Woodbury, MN (US); Russel A. Roiko, Rogers, MN (US); Eugene G. Joseph, Blacksburg, VA (US)

Correspondence Address: 3M INNOVATIVE PROPERTIES COMPANY, PO BOX 33427, ST. PAUL, MN, 55133-3427, US

(21) Appl. No.: 12/937,309

(22) PCT Filed: Apr. 16, 2009
(86) PCT No.: PCT/US09/40745

§ 371 Date: Oct. 11, 2010

Related U.S. Application Data
(60) Provisional application No. 61/046,063, filed on Apr. 18, 2008.

(51) Int. Cl. A61C 5/04; C08L 33/10; A61K 6/083

(52) U.S. Cl. 433/224; 525/55; 523/117; 523/116; 525/222

(57) ABSTRACT

Compositions comprising a hyperbranched compound and a polymer prepared from reactants comprising at least one (meth)acrylate monomer, and dental filling compositions comprising a hyperbranched compound.

### BACKGROUND

The practice of endodontics includes the treatment of diseased root canals, typically when a tooth is intact but the root or pulp tissue is diseased. Access to the root canal has been made by drilling an opening in a tooth surface. Subsequently, the root material has been removed from the root canal, and the canal has been enlarged and then filled.

Root canal filling materials have been made of natural rubbers, for example gutta percha. In some instances, the gutta percha filling materials have been placed, in the form of cylinders or cones, into root canals. The filling materials have then been compressed or heated. More recently, the gutta percha filling materials have been softened by heating using a “gun” which has then been used to force the filling material into the root canal. Root canal filling materials comprising gutta percha have been used in combination with dental or endodontic sealing materials to seal the root canal around the filling material.

### SUMMARY

There is a need for compositions for filling dental cavities, such as root canals, that have useful physical properties such as a low melting or softening temperature, sufficiently low viscosity when melted or softened to flow or be easily compacted into a root canal, and resistance to biological degradation.

In one aspect a composition is provided comprising a hyperbranched compound and a polymer prepared from reactants comprising at least one (meth)acrylate monomer.

In another aspect, a composition is provided comprising a hyperbranched polyester compound having a plurality of terminal alkyl ester groups, and a polymer prepared from reactants comprising at least one alkyl(meth)acrylate monomer having an alkyl group comprising at least six carbon atoms and at least one ethylenically unsaturated monomer having a polar group or a siloxane group.

In yet another aspect, a method of restoring a dental cavity is provided, the method comprising providing a composition comprising a hyperbranched compound, and inserting the composition into the dental cavity.

In yet another aspect, an article for filling a root canal is provided, comprising a hyperbranched compound, wherein the article has an aspect ratio of at least 2 to 1.

### DETAILED DESCRIPTION

In several places throughout the application, guidance is provided through lists of examples, which examples can be used in various combinations. In each instance, the recited list serves only as a representative group and should not be interpreted as an exclusive list.

Any recitation of numerical ranges by endpoints includes all numbers subsumed within that range (e.g., 1 to 5 includes 1, 1.5, 2, 2.75, 3, 3.80, 4, 5, etc.).

The terms “a,” “an,” “the,” “at least one,” and “one or more” are used interchangeably. Thus, for example, a composition that comprises “a” compound of Formula I can be interpreted to mean that the composition includes “one or more” compounds of Formula I.

The term “hyperbranched compound” refers to a hyperbranched polymer, a dendrimer or to a mixture of a hyperbranched polymer and a dendrimer.

The term “hyperbranched polymer” refers to a polymer having a main polymer chain and at least two branching points along the main polymer chain. At the branching points, branches (i.e., branch polymer chains) extend from the main polymer chain.

The term “dendrimer” refers to a compound having an arborescent (tree-like) structure of a core and branches. Typically, a dendrimer has a central core moiety and sequential branching beginning at the core moiety. A dendrimer often has a high degree of structural symmetry.

A composition is provided comprising a hyperbranched compound and a polymer prepared from reactants comprising at least one (meth)acrylate monomer.

The composition comprises at least one hyperbranched compound. The hyperbranched compound can comprise a hyperbranched polymer having a main polymer chain and at least two branching points along the main polymer chain. At the branching points, branches (i.e., branch polymer chains) extend from the main polymer chain. The branches of a hyperbranched polymer can themselves comprise branching points (i.e., additional branches can extend from the branches). A hyperbranched polymer can have any number of branching points on the main polymer chain or on the branches. The branching points can be regularly or irregularly spaced along the main polymer chain or along the branches. More than one branch can have the same molecular weight, or branches can independently have different molecular weights.

In some embodiments, the hyperbranched compound comprises a dendrimer. The dendrimer can comprise at least one organic core or organic branch. In some embodiments, the dendrimer comprises an inorganic core (e.g., a silica core) or inorganic branch. In other embodiments, the dendrimer comprises an organic core and an organic branch. The dendrimer can be free of an inorganic core. The dendrimer can be free of an inorganic branch. In some embodiments, the hyperbranched compound comprises less than 10 weight percent dendrimer, less than 5 weight percent dendrimer, less than 2 weight percent dendrimer, or less than 1 weight percent dendrimer. In some embodiments, the hyperbranched compound is free of dendrimer.

The composition can comprise a hyperbranched compound having a weight average molecular weight (in units of grams per mole) of at least 300, at least 500, at least 750, at least 1,000, at least 2,000, at least 3,000, at least 4,000, at least 5,000, at least 6,000, at least 7,000, at least 8,000, at least 9,000, at least 10,000, at least 12,000, at least 14,000, at least 16,000, at least 18,000, or at least 20,000. The composition can comprise a hyperbranched compound having a weight average molecular weight (in units of grams per mole) of no greater than 30,000, no greater than 28,000, no greater than 26,000, no greater than 24,000, no greater than 22,000, no greater than 20,000, no greater than 18,000, no greater than 16,000, no greater than 14,000, no greater than 12,000, no greater than 10,000, no greater than 9,000, no greater than 8,000, no greater than 7,000, no greater than 6,000, no greater than 5,000, or no greater than 4,000.

The composition can comprise a hyperbranched compound having a polydispersity index (PDI) of at least 1.00, at least 1.05, at least 1.10, at least 1.15, at least 1.20, at least 1.25, at least 1.30, at least 1.35, at least 1.40, at least 1.45, at least 1.50, at least 1.55, or at least 1.60. The composition can comprise a hyperbranched compound having a PDI of no greater than 1.10, no greater than 1.15, no greater than 1.20, no greater than 1.25, no greater than 1.30, no greater than 1.35, no greater than 1.40, no greater than 1.45, no greater than 1.50, no greater than 1.55, no greater than 1.60, no greater than 1.65, no greater than 1.70, no greater than 1.75, no greater than 1.80, no greater than 1.85, no greater than 1.90, no greater than 1.95, or no greater than 2.00.

The composition can comprise a hyperbranched compound having a glass transition temperature (T_{g}) of at least −100° C., at least −80° C., at least −70° C., at least −60° C., at least −50° C., at least −40° C., at least −30° C., at least −20° C., at least −10° C., at least 0° C., at least 10° C., at least 20° C., at least 30° C., or at least 40° C. The composition can comprise a hyperbranched compound having a glass transition temperature (T_{g}) of no greater than −70° C., no greater than −60° C., no greater than −50° C., no greater than −40° C., no greater than −30° C., no greater than −20° C., no greater than −10° C., no greater than 0° C., no greater than 10° C., no greater than 20° C., no greater than 30° C., no greater than 40° C., or no greater than 50° C.

The hyperbranched compound can comprise at least one hyperbranched polyether, hyperbranched polyester, hyperbranched polyamide, hyperbranched polyurea, hyperbranched polyurethane, or combinations thereof. For example, the hyperbranched compound can comprise at least one hyperbranched polyether, at least one hyperbranched polyester, at least one hyperbranched polyamide, at least one hyperbranched polyurea, or at least one hyperbranched polyurethane. Alternatively, the hyperbranched compound can comprise, for example, a hyperbranched polyester and a hyperbranched polyether, a hyperbranched polyester and a hyperbranched polyamide, or a hyperbranched polyurea and a hyperbranched polyurethane. In yet another alternative, the hyperbranched compound can comprise mixtures of more than two hyperbranched compounds (e.g., a hyperbranched polyamide, a hyperbranched polyurea, and a hyperbranched polyurethane).

A hyperbranched polymer can be a hyperbranched step-growth polymer prepared by, for example, condensation polymerization or cationic ring-opening polymerization. Hyperbranched aromatic or aliphatic polyesters can be prepared by condensation polymerization. For example, a hyperbranched polyester can be prepared from reactants comprising a polyfunctional carboxylic acid or a polyfunctional carboxylic acid ester and a polyfunctional alcohol. Hyperbranched aliphatic polyethers can be prepared by cationic ring opening polymerization of, for example, aliphatic compounds comprising an alcohol group and a cyclic ether group. The cyclic ether group can comprise, for example, an oxetane group.

The hyperbranched compound can comprise an aromatic hyperbranched polymer (i.e., a hyperbranched polymer comprising repeating units having an aromatic ring) or an aliphatic hyperbranched polymer (i.e., a hyperbranched polymer comprising repeating units having an aliphatic group). For example, in embodiments where the hyperbranched polymer comprises an aromatic hyperbranched polyester, the aromatic hyperbranched polyester can be prepared from reactants comprising at least one polyfunctional aromatic carboxylic acid, at least one polyfunctional aromatic carboxylic acid ester, or at least one polyfunctional aromatic alcohol. In embodiments where the hyperbranched polymer comprises an aliphatic hyperbranched polyester, the aliphatic hyperbranched polyester can be prepared from reactants comprising at least one polyfunctional aliphatic carboxylic acid or polyfunctional aliphatic carboxylic acid ester, and at least one polyfunctional aliphatic alcohol.

Typically, a hyperbranched compound is prepared from a reaction mixture comprising multifunctional reactants (i.e., reactants having more than two reactive functional groups). For example, a hyperbranched aliphatic polyester can be prepared from a reaction mixture comprising a trifunctional alcohol (e.g., trimethylolpropane) and an aliphatic carboxylic acid comprising two alcohol groups, as shown in Reaction Scheme A.

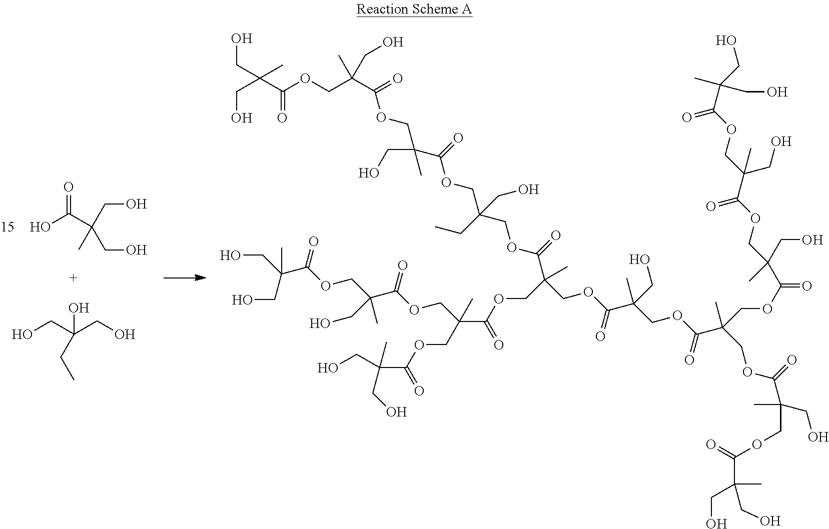

A hyperbranched compound typically comprises a plurality of terminal reactive functional groups (i.e., terminal reactive functional groups of the class of reactive function groups of at least one of the reactants). For example, the hyperbranched compound shown in Reaction Scheme A comprises a plurality of terminal hydroxyl groups. Non-limiting examples of terminal reactive functional groups include alcohol groups, primary amino groups, secondary amino groups, carboxylic acid groups, carbonyl halide groups, and carboxylic acid ester groups.

The terminal reactive functional groups of the hyperbranched compound can further react with a monofunctional compound to provide a hyperbranched compound comprising a modified terminal group. For example, a terminal alcohol group of a hyperbranched compound can react with a monofunctional carboxylic acid or a monofunctional carboxylic acid chloride to provide a hyperbranched compound comprising a terminal carboxylic acid ester group. In another example, a terminal alcohol group of a hyperbranched compound can react with a monofunctional isocyanate to provide a hyperbranched compound comprising a terminal urethane group. Non-limiting examples of modified terminal groups include alkyl ester groups, aromatic ester groups, alkyl ether groups, aromatic ether groups, alkyl amide groups, aromatic amide groups, alkyl urea groups, aromatic urea groups, alkyl urethane groups, and aromatic urethane groups. In some embodiments, the hyperbranched compound independently comprises at least one terminal ether, ester, amide, urea, or urethane group.

Alkyl terminal groups (in, for example, terminal alkyl ester groups or terminal alkyl ether groups) can independently comprise at least 1 carbon atom, at least 2 carbon atoms, at least 4 carbon atoms, at least 6 carbon atoms, at least 8 carbon atoms, at least 10 carbon atoms, at least 12 carbon atoms, at least 14 carbon atoms, at least 16 carbon atoms, at least 18 carbon atoms, at least 20 carbon atoms, or at least 22 carbon atoms. Alkyl terminal groups can independently comprise no greater than 4 carbon atoms, no greater than 6 carbon atoms, no greater than 8 carbon atoms, no greater than 10 carbon atoms, no greater than 12 carbon atoms, no greater than 14 carbon atoms, no greater than 16 carbon atoms, no greater than 18 carbon atoms, no greater than 20 carbon atoms, no greater than 22 carbon atoms, or no greater than 24 carbon atoms. Alkyl terminal groups can independently comprise linear, branched, or cyclic structures. Non-limiting examples of alkyl terminal groups include methyl, ethyl, propyl, isopropyl butyl, 2-butyl, pentyl, hexyl, octyl, decyl, dodecyl, tetratecyl, hexadecyl, octadecyl, eicosyl, and behenyl groups.

Aromatic terminal groups can independently comprise at least 4 carbon atoms, at least 6 carbon atoms, at least 8 carbon atoms, at least 10 carbon atoms, at least 12 carbon atoms, at least 14 carbon atoms, at least 16 carbon atoms, or at least 18 carbon atoms. Aromatic terminal groups can independently comprise no greater than 6 carbon atoms, no greater than 8 carbon atoms, no greater than 10 carbon atoms, no greater than 12 carbon atoms, no greater than 14 carbon atoms, no greater than 16 carbon atoms, no greater than 18 carbon atoms, or no greater than 20 carbon atoms. Non-limiting examples of aromatic terminal groups include unsubstituted phenyl and substituted phenyl.

One or more terminal reactive functional groups of the hyperbranched compound can react with a monofunctional compound to provide a hyperbranched compound comprising one or more modified terminal groups. At least 0.1 mole percent, at least 0.5 mole percent, at least 1 mole percent, at least 2 mole percent, at least 5 mole percent, at least 10 mole percent, at least 15 mole percent, at least 20 mole percent, at least 25 mole percent, at least 30 mole percent, at least 35 mole percent, at least 40 mole percent, at least 45 mole percent, at least 50 mole percent, at least 55 mole percent, at least 60 mole percent, at least 65 mole percent, at least 70 mole percent, at least 75 mole percent, at least 80 mole percent, at least 85 mole percent, at least 90 mole percent, or at least 95 mole percent of the terminal reactive functional groups of a hyperbranched compound can react with a monofunctional compound to provide a hyperbranched compound comprising one or more modified terminal groups. No greater than 0.5 mole percent, no greater than 1 mole percent, no greater than 2 mole percent, no greater than 5 mole percent, no greater than 10 mole percent, no greater than 15 mole percent, no greater than 20 mole percent, no greater than 25 mole percent, no greater than 30 mole percent, no greater than 35 mole percent, no greater than 40 mole percent, no greater than 45 mole percent, no greater than 50 mole percent, no greater than 55 mole percent, no greater than 60 mole percent, no greater than 65 mole percent, no greater than 70 mole percent, no greater than 75 mole percent, no greater than 80 mole percent, no greater than 85 mole percent, no greater than 90 mole percent, no greater than 95 mole percent, no greater than 96 mole percent, no greater than 98 mole percent, or no greater than 99 mole percent of the terminal reactive functional groups of a hyperbranched compound can react with a monofunctional compound to provide a hyperbranched compound comprising one or more modified terminal groups.

The hyperbranched compound can be substantially free of ethylenically unsaturated groups. The term “substantially free of ethylenically unsaturated groups” means that no greater than 1 mole percent, no greater than 0.5 mole percent, no greater than 0.2 mole percent, no greater than 0.1 mole percent, no greater than 0.05 mole percent, no greater than 0.01 mole percent, no greater than 0.005 mole percent, or no greater than 0.001 mole percent of any functional group of the hyperbranched compound comprise terminal groups comprising ethylenically unsaturated groups. In some embodiments, the hyperbranched compound is free of ethylenically unsaturated groups.

The hyperbranched compound can comprise a crystalline hyperbranched compound (i.e., a hyperbranched compound having a crystalline melting point as measure by, for example, differential scanning calorimetry (DSC)). The crystalline hyperbranched compound can comprise a hyperbranched polymer having a crystalline main polymer chain, a crystalline branch, or both. The crystalline hyperbranched compound can have a crystalline melting point of at least −40° C., at least −30° C., at least −20° C., at least −10° C., at least 0° C., at least 10° C., at least 20° C., at least 30° C., at least 40° C., or at least 50° C. The crystalline hyperbranched compound can have a crystalline melting point of no greater than −20° C., no greater than −10° C., no greater than 0° C., no greater than 10° C., no greater than 20° C., no greater than 30° C., no greater than 40° C., no greater than 50° C., no greater than 60° C., or no greater than 70° C.

In some embodiments, the hyperbranched compound is substantially free of a crystalline hyperbranched compound (i.e., the hyperbranched compound comprises less than 5 mole percent, less than 2 mole percent, less than 1 mole percent, or less than 0.5 mole percent crystalline hyperbranched compound). In other embodiments, the hyperbranched compound can comprise a hyperbranched compound that is free of a crystalline hyperbranched compound (i.e., the hyperbranched compound is amorphous).

The hyperbranched compound can have a softening or melting temperature no greater than 0° C., no greater than 10° C., no greater than 20° C., no greater than 30° C., no greater than 40° C., no greater than 50° C., no greater than 60° C., or no greater than 70° C. The hyperbranched compound can have a softening or melting temperature of at least 10° C., at least 20° C., at least 30° C., at least 40° C., at least 50° C., at least 60° C., at least 70° C., or at least 80° C. The term “softening temperature” refers to the temperature at which a hyperbranched compound (in the form of free-flowing pellets or powder) no longer flows freely. Alternatively, the term “softening temperature” refers to the temperature at which a hyperbranched compound (in the form of, for example, a cylinder or a sheet) begins to deform (e.g., sag under its own weight) under the force of gravity.

In addition to the hyperbranched compound, the composition further comprises a polymer prepared from reactants comprising at least one (meth)acrylate monomer. The polymer can be prepared from reactants comprising at least one (meth)acrylate monomer and at least one ethylenically unsaturated monomer having a polar group or a siloxane group. The (meth)acrylate monomer can comprise an alkyl, aryl, or aralkyl (meth)acrylate monomer.

The (meth)acrylate monomer can comprise a compound of Formula I

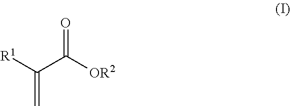

wherein R¹ comprises a hydrogen atom or an alkyl group having 1 to 4 carbon atoms, and R² comprises an alkyl, aryl, or aralkyl group having no greater than 30 carbon atoms.

In some embodiments, R¹ is a hydrogen atom (i.e., the (meth)acylate monomer comprises an acrylate monomer). In other embodiments, R¹ is an alkyl group having 1 to 4 carbon atoms. When R¹ is an alkyl group, the alkyl group can comprise a linear or branched structure. For example, R¹ can comprise a methyl group (i.e., the (meth)acrylate monomer comprises a methacrylate monomer), an ethyl group, an n-propyl group, an isopropyl group, an n-butyl group, or an isobutyl group.

In some embodiments, R² comprises an alkyl group. The alkyl group can comprise linear, branched, or cyclic structures. The alkyl group can comprise no greater than 30 carbon atoms, no greater than 28 carbon atoms, no greater than 26 carbon atoms, no greater than 24 carbon atoms, no greater than 22 carbon atoms, no greater than 20 carbon atoms, no greater than 18 carbon atoms, no greater than 16 carbon atoms, no greater than 14 carbon atoms, no greater than 12 carbon atoms, no greater than 10 carbon atoms, no greater than 8 carbon atoms, no greater than 6 carbon atoms, no greater than 4 carbon atoms, no greater than 2 carbon atoms, or 1 carbon atom. The alkyl group can comprise at least 26 carbon atoms, at least 24 carbon atoms, at least 22 carbon atoms, at least 20 carbon atoms, at least 18 carbon atoms, at least 16 carbon atoms, at least 14 carbon atoms, at least 12 carbon atoms, at least 10 carbon atoms, at least 8 carbon atoms, at least 6 carbon atoms, or at least 4 carbon atoms. Non-limiting examples of alkyl groups include methyl, ethyl, propyl, butyl, hexyl, octyl, decyl, dodecyl(lauryl), tetradecyl, hexadecyl, octadecyl, eicosyl, docosyl, tetracosyl, hexacosyl, octacosyl, triacontyl, 2-propyl, 2-butyl, 2-hexyl, 3-octyl, 2-decyl, 4-dodecyl, cyclohexyl, and cyclohexylmethyl.

In some embodiments, the (meth)acrylate monomer comprises an alkyl(meth)acrylate monomer. In some embodiments, the alkyl(meth)acrylate monomer comprises a compound of Formula I wherein R¹ comprises a hydrogen atom or a methyl group, and R² comprises an alkyl group having 8 to 24 carbon atoms. In some embodiments, the (meth)acrylate monomer comprises isobornyl acrylate, isobornyl methacrylate, dodecyl acrylate(lauryl acrylate), dodecyl methacrylate(lauryl methacrylate), tetradecyl acrylate, tetradecyl methacrylate, hexadecyl acrylate, hexadecyl methacrylate, octadecyl acrylate, octadecyl methacrylate, behenyl acrylate, or behenyl methacrylate.

In some embodiments, R² comprises an aryl group. The aryl group can comprise one arene ring or more than one arene ring. Aryl groups can comprise up to 6 carbon atoms, up to 8 carbon atoms, up to 10 carbon atoms, up to 12 carbon atoms, up to 14 carbon atoms, up to 16 carbon atoms, or up to 18 carbon atoms. If more than one arene ring is present in an aryl group, the arene rings can be fused together, or they can be joined by a chemical bond. Non-limiting examples of aryl groups include substituted and unsubstituted phenyl, 1-naphthyl, 2-naphthyl, 9-anthracenyl, and biphenyl.

In some embodiments, R² comprises an aralkyl group. The aralkyl group can comprise one arene ring or more than one arene ring. The aralkyl group can comprise up to 6 carbon atoms, up to 8 carbon atoms, up to 10 carbon atoms, up to 12 carbon atoms, up to 14 carbon atoms, up to 16 carbon atoms, up to 18 carbon atoms, or up to 20 carbon atoms. If more than one arene ring is present in the aralkyl group, the arene rings can be fused together, or they can be joined by a chemical bond. The aralkyl group can comprise one or more alkyl groups. The alkyl group can comprise linear, branched, or cyclic structures. The alkyl groups can be bonded to an arene ring, and can comprise no greater than 24 carbon atoms, no greater than 22 carbon atoms, no greater than 20 carbon atoms, no greater than 18 carbon atoms, no greater than 16 carbon atoms, no greater than 14 carbon atoms, no greater than 12 carbon atoms, no greater than 10 carbon atoms, no greater than 8 carbon atoms, no greater than 6 carbon atoms, or no greater than 4 carbon atoms. The alkyl group can comprise at least 18 carbon atoms, at least 16 carbon atoms, at least 14 carbon atoms, at least 12 carbon atoms, at least 10 carbon atoms, at least 8 carbon atoms, at least 6 carbon atoms, at least 4 carbon atoms, at least 2 carbon atoms, or at least 1 carbon atom. Examples of alkyl groups include methyl, ethyl, 1-propyl, 2-propyl, 1-butyl, and 2-butyl groups. Non-limiting examples of aralkyl groups include benzyl, 4-methyl benzyl, 1-phenylethyl, 2-phenylethyl, 3-phenylpropyl, 2-naphthylethyl, and 9-anthracenylmethyl.

The ethylenically unsaturated monomer comprising a polar group can comprise a compound of Formula II, Formula III, or Formula IV

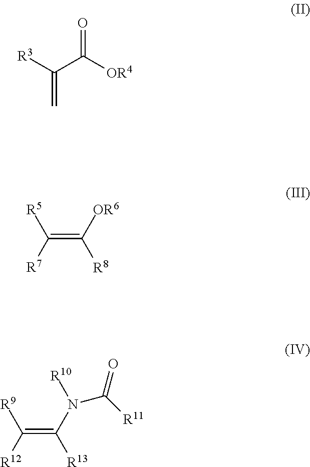

wherein R³, R⁵, R⁷, R⁸, R⁹, R¹², and R¹³ independently can comprise a hydrogen atom or an alkyl group having 1 to 4 carbon atoms. In Formula II, R⁴ can comprise a substituted or unsubstituted heteroalkyl group having 1 to 400 carbon atoms. In Formula III, R⁶ can comprise 1 to 20 carbon atoms. In Formula IV, R¹⁰ can comprise a hydrogen atom or an alkyl group having 1 to 8 carbon atoms (the alkyl group optionally substituted with a carbonyl group), and R¹¹ can comprise an alkyl group having 1 to 8 carbon atoms. Alternatively, in some embodiments R¹⁰ and R¹¹ can together form a ring structure including the nitrogen atom.

In Formulas II, III, and IV, the groups R³, R⁵, R⁷, R⁸, R⁹, R¹², and R¹³ independently comprise a hydrogen atom or an alkyl group having 1 to 4 carbon atoms. When R³, R⁵, R⁷, R⁸, R⁹, R¹², and R¹³ independently comprise an alkyl group, the alkyl group can comprise a linear or branched structure. For example, R³, R⁵, R⁷, R⁸, R⁹, R¹², and R¹³ can independently be a methyl group, an ethyl group, an n-propyl group, an isopropyl group, an n-butyl group, or an isobutyl group.

In Formula II, R⁴ can comprise a substituted or unsubstituted heteroalkyl group having 1 to 400 carbon atoms. Often, R⁴ comprises a substituted or unsubstituted heteroalkyl group having no greater than 30 carbon atoms. The heteroalkyl group (i.e., an alkyl group that comprises at least one heteroatom, e.g., oxygen, nitrogen, or sulfur) can comprise a linear, branched, or cyclic structure. The heteroalkyl group can comprise no greater than 30 carbon atoms, no greater than 28 carbon atoms, no greater than 26 carbon atoms, no greater than 24 carbon atoms, no greater than 22 carbon atoms, no greater than 20 carbon atoms, no greater than 18 carbon atoms, no greater than 16 carbon atoms, no greater than 14 carbon atoms, no greater than 12 carbon atoms, no greater than 10 carbon atoms, no greater than 8 carbon atoms, no greater than 6 carbon atoms, or no greater than 4 carbon atoms. The heteroalkyl group can comprise at least 18 carbon atoms, at least 16 carbon atoms, at least 14 carbon atoms, at least 12 carbon atoms, at least 10 carbon atoms, at least 8 carbon atoms, at least 6 carbon atoms, at least 4 carbon atoms, at least 2 carbon atoms, or at least 1 carbon atom. The heteroalkyl group can comprise no greater than 30 heteroatoms, no greater than 28 heteroatoms, no greater than 26 heteroatoms, no greater than 24 heteroatoms, no greater than 22 heteroatoms, no greater than 20 heteroatoms, no greater than 18 heteroatoms, no greater than 16 heteroatoms, no greater than 14 heteroatoms, no greater than 12 heteroatoms, no greater than 10 heteroatoms, no greater than 8 heteroatoms, no greater than 6 heteroatoms, or no greater than 4 heteroatoms. The heteroalkyl group can comprise at least 24 heteroatoms, at least 22 heteroatoms, at least 20 heteroatoms, at least 18 heteroatoms, at least 16 heteroatoms, at least 14 heteroatoms, at least 12 heteroatoms, at least 10 heteroatoms, at least 8 heteroatoms, at least 6 heteroatoms, at least 4 heteroatoms, at least 2 heteroatoms, or at least 1 heteroatom.

Non-limiting examples of heteroalkyl groups include amino groups such as 3-N,N-dimethylaminopropyl, ether groups such as methoxyethyl, and polyether groups (i.e., a group comprising more than one ether group) such as methoxyethoxyethyl and tetrahydrofurfuryl. Ether and polyether groups can comprise oxyalkylene groups, for example groups having the structure of Formula V

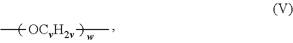

where v is an integer of 1 to 4 and w is an integer of 1 to 100. An ether group can include a group of Formula V where w is 1. Non-limiting examples of polyether groups comprising oxyalkylene groups include poly(oxymethylene), poly(oxyethylene), and poly(oxybutylene) groups. In Formula V, w can be an integer of at least 1, at least 2, at least 4, at least 6, at least 8, at least 10, at least 20, at least 30, at least 40, at least 50, at least 60, at least 80, or at least 90. In Formula V, w can be an integer of 100, no greater than 100, no greater than 80, no greater than 60, no greater than 50, no greater than 40, no greater than 20, no greater than 10, no greater than 8, no greater than 6, no greater than 4, or no greater than 2.

In Formula III, the group R⁶ can comprise 1 to 20 carbon atoms. The group R⁶ can comprise at least 1 carbon atom, at least 2, at least 3, at least 4, at least 5, at least 6, at least 7, at least 8, at least 9, at least 10, at least 12, at least 14, or at least 16 carbon atoms. The group R⁶ can comprise no greater than 20, no greater than 18, no greater than 16, no greater than 14, no greater than 12, no greater than 10, no greater than 9, no greater than 8, no greater than 7, no greater than 6, no greater than 5, no greater than 4, or no greater than 3 carbon atoms.

In some embodiments, R⁶ comprises an alkyl group (optionally substituted with a carbonyl group). In embodiments wherein R⁶ comprises an alkyl group, the compounds of Formula III can comprise an alkyl vinyl ether. Non-limiting examples of alkyl vinyl ethers include methyl vinyl ether and ethyl vinyl ether. In embodiments wherein the alkyl group is substituted with a carbonyl group, the compounds of Formula III can comprise a vinyl ester. Non-limiting examples of vinyl esters include vinyl acetate and vinyl propionate.

In some embodiments, R⁶ comprises a heteroalkyl group. The heteroalkyl group (i.e., an alkyl group that comprises at least one heteroatom, e.g., oxygen, nitrogen, or sulfur) can comprise a linear, branched, or cyclic structure. The heteroalkyl group can comprise no greater than 20 carbon atoms, no greater than 18 carbon atoms, no greater than 16 carbon atoms, no greater than 14 carbon atoms, no greater than 12 carbon atoms, no greater than 10 carbon atoms, no greater than 9 carbon atoms, no greater than 8 carbon atoms, no greater than 7 carbon atoms, no greater than 6 carbon atoms, no greater than 5 carbon atoms, or no greater than 4 carbon atoms. The heteroalkyl group can comprise at least 14 carbon atoms, at least 12 carbon atoms, at least 10 carbon atoms, at least 9 carbon atoms, at least 8 carbon atoms, at least 7 carbon atoms, at least 6 carbon atoms, at least 5 carbon atoms, at least 4 carbon atoms, at least 3 carbon atoms, at least 2 carbon atoms, or at least 1 carbon atom. The heteroalkyl group can comprise no greater than 20 heteroatoms, no greater than 18 heteroatoms, no greater than 16 heteroatoms, no greater than 14 heteroatoms, no greater than 12 heteroatoms, no greater than 10 heteroatoms, no greater than 9 heteroatoms, no greater than 8 heteroatoms, no greater than 7 heteroatoms, no greater than 6 heteroatoms, no greater than 5 heteroatoms, or no greater than 4 heteroatoms. The heteroalkyl group can comprise at least 16 heteroatoms, at least 14 heteroatoms, at least 12 heteroatoms, at least 10 heteroatoms, at least 9 heteroatoms, at least 8 heteroatoms, at least 7 heteroatoms, at least 6 heteroatoms, at least 5 heteroatoms, at least 4 heteroatoms, at least 3 heteroatoms, at least 2 heteroatoms, or at least 1 heteroatom. Non-limiting examples of heteroalkyl groups include amino groups such as 3-N,N-dimethylaminopropyl, ether groups such as methoxyethyl, and polyether groups (i.e., a group comprising more than one ether group) such as methoxyethoxyethyl and tetrahydrofurfuryl. Ether and polyether groups can comprise oxyalkylene groups, for example groups having the structure of Formula IV wherein v is an integer of 1 to 4 and w is an integer of 1 to 20.

In some embodiments, R⁶ comprises an aryl group. The aryl group can comprise at least 4 carbon atoms, at least 5 carbon atoms, at least 6 carbon atoms, at least 7 carbon atoms, at least 8 carbon atoms, at least 9 carbon atoms, or at least 10 carbon atoms. The aryl group can comprise no greater than 14 carbon atoms, no greater than 13 carbon atoms, no greater than 12 carbon atoms, no greater than 11 carbon atoms, no greater than 10 carbon atoms, no greater than 9 carbon atoms, no greater than 8 carbon atoms, no greater than 7 carbon atoms, or no greater than 6 carbon atoms. Non-limiting examples of aryl groups include phenyl, 1-naphthyl, 2-naphthyl, and 9-anthracenyl.

In some embodiments, R⁶ comprises an aralkyl group (optionally substituted with a carbonyl group). The aralkyl group can comprise at least 4 carbon atoms, at least 5 carbon atoms, at least 6 carbon atoms, at least 7 carbon atoms, at least 8 carbon atoms, at least 9 carbon atoms, at least 10 carbon atoms, at least 11 carbon atoms, at least 12 carbon atoms, at least 13 carbon atoms, or at least 14 carbon atoms. The aralkyl group can comprise no greater than 16 carbon atoms, no greater than14 carbon atoms, no greater than 13 carbon atoms, no greater than 12 carbon atoms, no greater than 11 carbon atoms, no greater than 10 carbon atoms, no greater than 9 carbon atoms, no greater than 8 carbon atoms, no greater than 7 carbon atoms, or no greater than 6 carbon atoms. Non-limiting examples of aralkyl groups include benzyl, 4-methyl benzyl, 1-phenylethyl, 2-phenylethyl, 3-phenylpropyl, 2-naphthylethyl, and 9-anthracenylmethyl.

In Formula IV, R¹⁰ can comprise a hydrogen atom or an alkyl group having 1 to 8 carbon atoms (the alkyl group optionally substituted with a carbonyl group), and R¹¹ can comprise an alkyl group having 1 to 8 carbon atoms. In some embodiments, R¹⁰ comprises a hydrogen atom. Alternatively, the group R¹⁰ can comprise an alkyl group having at least 1, at least 2, at least 3, at least 4, at least 5, at least 6, at least 7, or at least 8 carbon atoms. The group R¹⁰ can comprise an alkyl group having no greater than 3, no greater than 4, no greater than 5, no greater than 6, no greater than 7, no greater than 8, or no greater than 10 carbon atoms. When R¹° comprises an alkyl group having 1 to 8 carbon atoms, the compounds of Formula IV can be N-alkyl-N-vinyl carboxamide compounds. Non-limiting example of such compounds include N-methyl-N-vinyl acetamide and N-vinyl acetamide.

In some embodiments, R¹⁰ comprises an alkyl group substituted with a carbonyl group. The carbonyl group can be bonded (via a covalent bond) to the nitrogen atom. In embodiments wherein R¹⁰ comprises an alkyl group substituted with a carbonyl group that is bonded to the nitrogen atom, the compounds of Formula IV can be N-vinyl carboximide compounds.

In some embodiments, R¹⁰ and R¹¹ can together form a ring structure including the nitrogen atom. When R¹⁰ and R¹¹ together form a ring structure including the nitrogen atom, the ring structure comprises a N-vinyl cyclic carboxamide or (in the case where R¹⁰ comprises an alkyl group substituted with a carbonyl group) a N-vinyl cyclic carboximide. Non-limiting examples of N-vinyl cyclic carboxamides include N-vinyl pyrrolidinone and N-vinyl caprolactam. Non-limiting examples of N-vinyl cyclic carboximides include N-vinyl succinimide and N-vinyl glutarimide.

The ethylenically unsaturated monomer comprising a siloxane group can comprise a compound of Formula VI

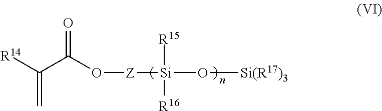

wherein R¹⁴ comprises a hydrogen atom or an alkyl group having 1 to 4 carbon atoms, Z is a divalent linking group, R¹⁵, R¹⁶, and R¹⁷ are independently alkyl groups, aryl groups, or aralkyl groups, and n is an integer of at least 1.

In Formula VI, R¹⁴ can, in some embodiments, comprise a hydrogen atom. In other embodiments, R¹⁴ comprises an alkyl group having 1 to 4 carbon atoms. When R¹⁴ is an alkyl group, the alkyl group can comprise a linear or branched structure. For example, R¹⁴ can comprise a methyl group, an ethyl group, an n-propyl group, an isopropyl group, an n-butyl group, or an isobutyl group.

The divalent linking group Z can be any divalent group. In some embodiments, the divalent linking group Z comprises at least one carbon atom bonded via a covalent bond to the silicon atom. Non-limiting examples of divalent linking groups include alkylene groups (e.g., ethylene or propylene groups), and arylene groups (e.g., a phenylene group). The alkylene groups can comprise a linear, branched, or cyclic structure. The divalent linking group Z can comprise 1 to 20 carbon atoms and can optionally include, for example, one or more ester, amide, urea, or urethane groups.

In Formula VI, R¹⁵, R¹⁶, and R¹⁷ are independently alkyl groups, aryl groups, or aralkyl groups. The alkyl group can comprise linear, branched, or cyclic structures. The alkyl group can comprise no greater than 10 carbon atoms, no greater than 8 carbon atoms, no greater than 6 carbon atoms, no greater than 4 carbon atoms, or no greater than 2 carbon atoms. The alkyl group can comprise at least 8 carbon atoms, at least 6 carbon atoms, at least 4 carbon atoms, at least 2 carbon atoms, or at least 1 carbon atom. Non-limiting examples of alkyl groups include methyl, ethyl, propyl, butyl, hexyl, octyl, 2-propyl, 2-butyl, 2-hexyl, 3-octyl, cyclohexyl, and cyclohexylmethyl.

In Formula VI, n is an integer of at least 1, at least 2, at least 5, at least 10, at least 20, at least 30, at least 40, at least 50, at least 60, at least 70, at least 80, at least 90, or at least 100. In Formula VI, n is an integer of no greater than 2, no greater than 5, no greater than 10, no greater than 20, no greater than 30, no greater than 40, no greater than 50, no greater than 60, no greater than 70, or no greater than 80.

In some embodiments, R¹⁵, R¹⁶, and R¹⁷ independently comprise a substituted or unsubstituted aryl group. The aryl group can comprise one arene ring or more than one arene ring. Aryl groups can comprise up to 6 carbon atoms, up to 8 carbon atoms, up to 10 carbon atoms, up to 12 carbon atoms, or up to 14 carbon atoms. If more than one arene ring is present in an aryl group, the arene rings can be fused together, or they can be joined by a chemical bond. Non-limiting examples of aryl groups include substituted and unsubstituted phenyl, 4-methylphenyl, 1-naphthyl, 2-naphthyl, 9-anthracenyl, and biphenyl.

In some embodiments, R¹⁵, R¹⁶, and R¹⁷ independently comprise a substituted or unsubstituted aralkyl group. The aralkyl group can comprise one arene ring or more than one arene ring. The aralkyl group can comprise up to 6 carbon atoms, up to 8 carbon atoms, up to 10 carbon atoms, up to 12 carbon atoms, up to 14 carbon atoms, up to 16 carbon atoms, up to 18 carbon atoms, or up to 20 carbon atoms. If more than one arene ring is present in the aralkyl group, the arene rings can be fused together, or they can be joined by a chemical bond. Non-limiting examples of aralkyl groups include benzyl, 4-methyl benzyl, 1-phenylethyl, 2-phenylethyl, 3-phenylpropyl, 2-naphthylethyl, and 9-anthracenylmethyl.

Representative examples of compounds of Formula VI include, for example, methacryloxypropyl-terminated poly(dimethylsiloxane).

The polymer can have a weight average molecular weight of at least 5,000, at least 10,000, at least 25,000, at least 50,000, at least 75,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, or at least 800,000. The polymer can have a weight average molecular weight of no greater than 10,000, no greater than 20,000, no greater than 25,000, no greater than 50,000, no greater than 75,000, no greater than 100,000, no greater than 150,000, no greater than 200,000, no greater than 250,000, no greater than 300,000, no greater than 350,000, no greater than 400,000, no greater than 450,000, no greater than 500,000, no greater than 550,000, no greater than 600,000, no greater than 650,000, no greater than 700,000, no greater than 750,000, no greater than 800,000, no greater than 850,000, no greater than 900,000, no greater than 950,000, or no greater than 1,000,000.

The polymer can have a glass transition temperature (T_{g}) of at least −100° C., at least −80° C., at least −70° C., at least −60° C., at least −50° C., at least −40° C., at least −30° C., at least −20° C., at least −10° C., at least 0° C., at least 10° C., at least 20° C., at least 30° C., at least 40° C., or at least 50° C. The polymer can have a glass transition temperature (T_{g}) of no greater than −80° C., no greater than −70° C., no greater than −60° C., no greater than −50° C., no greater than −40° C., no greater than −30° C., no greater than −20° C., no greater than −10° C., no greater than 0° C., no greater than 10° C., no greater than 20° C., no greater than 30° C., no greater than 40° C., no greater than 50° C., or no greater than 60° C.

In some embodiments, the polymer is a pressure sensitive adhesive. In this context, the term “pressure sensitive adhesive” refers to a polymer (or to a composition comprising a polymer) with properties including aggressive and persistent tack, adherence with no more than finger pressure, sufficient ability to hold onto an adherent, sufficient cohesive strength, and no activation by an energy source. Pressure sensitive adhesives can be tacky at temperatures at or above room temperature (i.e., at or above about 10° C. to about 30° C. or greater).

In some embodiments, the polymer comprises a linear polymer, i.e., a polymer comprising a linear polymer chain structure. In some embodiments, the polymer comprises a branched structure. In some embodiments, the polymer is substantially free of branching (i.e., the polymer comprises polymer chains having no greater than one branching point along the main polymer chain). Typically, the polymer is free of core/shell structure (i.e., the polymer does not comprise a core/shell polymer).

The polymer can be crosslinked. In some embodiments, the polymer is substantially free of crosslinks, i.e., the polymer has no greater than 5 mole percent, no greater than 2 mole percent, no greater than 1 mole percent, no greater than 0.5 mole percent, no greater than 0.2 mole percent, no greater than 0.1 mole percent, no greater than 0.05 mole percent, no greater than 0.02 mole percent, or no greater than 0.01 mole percent crosslinks (formed by reaction of a cure site on the polymer chain or by reaction of a crosslinking agent). In still other embodiments, the polymer is free of crosslinks.

The composition can comprise any weight percentage of the hyperbranched compound, based on the combined weights of the hyperbranched compound and the polymer. The composition can comprise at least 1 weight percent, at least 2 weight percent, at least 5 weight percent, at least 10 weight percent, at least 20 weight percent, at least 30 weight percent, at least 40 weight percent, at least 50 weight percent, at least 60 weight percent, or at least 70 weight percent of the hyperbranched compound, based on the combined weights of the hyperbranched compound and the polymer. The composition can comprise no greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent of the hyperbranched compound, based on the combined weights of the hyperbranched compound and the polymer. The composition can comprise one hyperbranched compound or more than one hyperbranched compound.

The composition can comprise any weight percentage of the polymer, based on the combined weights of the hyperbranched compound and the polymer. The composition can comprise at least 1 weight percent, at least 2 weight percent, at least 5 weight percent, at least 10 weight percent, at least 20 weight percent, at least 30 weight percent, at least 40 weight percent, at least 50 weight percent, at least 60 weight percent, or at least 70 weight percent of the polymer, based on the combined weights of the hyperbranched compound and the polymer. The composition can comprise no greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent of the polymer, based on the combined weights of the hyperbranched compound and the polymer. The composition can comprise one polymer or more than one polymer.

The hyperbranched compound and the polymer can be compatible. In this context, the term “compatible” refers to a tendency of a mixture of the hyperbranched compound and the polymer to be macroscopically homogeneous. That is, the mixture appears to be homogeneous (i.e., a single phase) when observed using the unaided eye. In some embodiments, the mixture appears to be homogeneous when observed using an optical microscope. In other embodiments, the mixture appears to be homogeneous when observed using an electron microscope.

The hyperbranched compound can dissolve in the polymer to form a solution of the hyperbranched compound in the polymer. At least 5 weight percent, at least 10 weight percent, at least 20 weight percent, at least 30 weight percent, at least 40 weight percent, at least 50 weight percent, at least 60 weight percent, at least 70 weight percent, or at least at least 80 weight percent of the hyperbranched compound can dissolve in the polymer in the composition. No greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent of the hyperbranched compound can dissolve in the polymer in the composition.

The polymer can dissolve in the hyperbranched compound to form a solution of the polymer in the hyperbranched compound. At least 5 weight percent, at least 10 weight percent, at least 20 weight percent, at least 30 weight percent, at least 40 weight percent, at least 50 weight percent, at least 60 weight percent, or at least 70 weight percent of the polymer can dissolve in the hyperbranched compound in the composition. No greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent of the polymer can dissolve in the hyperbranched compound in the composition. In some embodiments, the hyperbranched compound and the polymer are miscible.

The hyperbranched compound and the polymer can react with each other to form, for example, hydrogen bonds, ionic bonds, or covalent bonds. Hydrogen, ionic or covalent bonds between the hyperbranched compound and the polymer can form a crosslinked network wherein the hyperbranched compound is bonded to the polymer via more than one hydrogen, ionic, or covalent bond. Typically, the hyperbranched compound and the polymer do not react with each other to form, for example, hydrogen, ionic, or covalent bonds. In some embodiments, the hyperbranched compound comprises organic functional groups that are capable of reacting with organic functional groups on the polymer to form hydrogen, ionic, or covalent bonds, but these functional groups typically do not react with each other under conditions of, for example, temperatures reached during processing or use of the compositions. In some embodiments, the composition is substantially free of hydrogen, ionic, or covalent bonds between the hyperbranched compound and the polymer. The term “substantially free of hydrogen, ionic, or covalent bonds” refers to a composition in which at least one of the hyperbranched compound or the polymer can be dissolved in a solvent to form a solution of at least one of the hyperbranched compound or the polymer in the solvent. In some embodiments, the composition is free of hydrogen, ionic, or covalent bonds between the hyperbranched compound and the polymer.

The composition can comprise a crosslinking agent. A crosslinking agent can link together (i.e., can form covalent bonds with), for example, each of at least two polymer chains or at least one polymer chain and one hyperbranched compound. The crosslinking agent can be, for example, a di- or polyfunctional ethylenically unsaturated monomer, for example, a di- or polyfunctional (meth)acrylate monomer. In some embodiments, the composition comprises less than 10 weight percent crosslinking agent, based on the combined weights of the hyperbranched compound and the polymer. In some embodiments, the composition is substantially free of crosslinking agent, i.e., it comprises less than 8 weight percent, less than 6 weight percent, less than 4 weight percent, less than 2 weight percent, less than 1 weight percent, less than 0.5 weight percent, less than 0.2 weight percent, less than 0.1 weight percent, or less than 0.05 weight percent crosslinking agent, based on the combined weights of the hyperbranched compound and the polymer. In some embodiments, the composition is free of crosslinking agent.

The composition can be substantially free of ethylenically unsaturated groups. The term “substantially free of ethylenically unsaturated groups” means that no greater than 1 mole percent, no greater than 0.5 mole percent, no greater than 0.2 mole percent, no greater than 0.1 mole percent, no greater than 0.05 mole percent, no greater than 0.01 mole percent, no greater than 0.005 mole percent, or no greater than 0.001 mole percent of the functional groups of any component of the composition comprises ethylenically unsaturated groups. In some embodiments, the composition is free of ethylenically unsaturated groups.

The composition can comprise additional components such as fillers, dyes, pigments, flavoring agents, or medicaments such as anticaries agents (e.g., fluoride sources) or antibiotics.

The composition can comprise a polyterpene such as gutta percha. In some embodiments, the composition is substantially free of gutta percha. In this context. “substantially free of gutta percha” refers to a composition comprising less than 15 weight percent, less than 10 weight percent, less than 5 weight percent, less than 2 weight percent, less than 1 weight percent, or less than 0.5 weight percent gutta percha. In some embodiments, the composition is free of gutta percha.

The composition can comprise at least one filler. A filler can be an inorganic filler comprising oxides of silicon (silicas) or oxides of zirconium (zirconias), and can further comprise oxides of other chemical elements such yttrium. Suitable silicas include fumed silica and nanoparticulate silica. Suitable zirconias include nanoparticulate zirconias. In some embodiments, the fillers are surface-modified inorganic fillers (i.e., inorganic fillers modified with organic groups). Suitable inorganic fillers are described in, for example, U.S. Patent Application Publication No. 2005/0256223 (Kolb, et al.) and U.S. Pat. No. 6,387,981 (Zhang et al.), U.S. Pat. No. 6,572,693 (Wu et al.), U.S. Pat. No. 7,090,721 (Craig et al.), and U.S. Pat. No. 7,156,911 (Kangas et al.).

The filler can have any particle size. In some embodiments, the filler is agglomerated (i.e., the primary filler particles have formed clusters or clumps). In some embodiments, the filler is not agglomerated (i.e., the filler can be substantially free of agglomerated primary particles). The filler primary particle size can be any particles size. In some embodiments, the primary particle size is at least 40 micrometers, at least 30 micrometers, at least 20 micrometers, at least 10 micrometers, at least 5 micrometers, at least 2 micrometers, at least 1 micrometers, at least 800 nanometers, at least 600 nanometers, at least 400 nanometers, at least 200 nanometers, at least 100 nanometers, at least 50 nanometers, at least 25 nanometers, at least 10 nanometers, at least 5 nanometers, at least 2 nanometers, or at least 1 nanometer. In some embodiments, the primary particle size is no greater than 60 micrometers, no greater than 50 nanometers, no greater than 40 micrometers, no greater than 30 micrometers, no greater than 20 micrometers, no greater than 10 micrometers, no greater than 5 micrometers, no greater than 2 micrometers, no greater than 1 micrometers, no greater than 800 nanometers, no greater than 600 nanometers, no greater than 400 nanometers, no greater than 200 nanometers, no greater than 100 nanometers, no greater than 50 nanometers, no greater than 25 nanometers, no greater than 10 nanometers, or no greater than 5 nanometers.

The composition can comprise at least 1 weight percent, at least 2 weight percent, at least 5 weight percent, at least 10 weight percent, at least 15 weight percent, at least 20 weight percent, at least 25 weight percent, at least 30 weight percent, at least 35 weight percent, at least 40 weight percent, at least 45 weight percent, at least 50 weight percent, at least 55 weight percent, at least 60 weight percent, at least 65 weight percent, or at least 70 weight percent inorganic filler, based on the total weight of the composition. The composition can comprise no greater than 85 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent inorganic filler, based on the total weight of the composition.

In some embodiments, the fillers comprise radiopaque inorganic fillers such as various barium compounds (e.g., barium sulfate, barium ziconate, barium strontium titanium oxide, or barium tungstate) or oxides of zirconium (including yttrium-containing oxides of zirconium). The fillers can comprise at least 1 weight percent, at least 2 weight percent, at least 5 weight percent, at least 10 weight percent, at least 15 weight percent, at least 20 weight percent, at least 25 weight percent, at least 30 weight percent, at least 35 weight percent, at least 40 weight percent, at least 45 weight percent, at least 50 weight percent, at least 55 weight percent, at least 60 weight percent, at least 65 weight percent, at least 70 weight percent, at least 75 weight percent, or at least 80 weight percent percent radiopaque filler, based on the total weight of the filler in the composition. The fillers can comprise no greater than 99 weight percent, no greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent radiopaque filler, based on the total weight of the filler in the composition.

The composition can further comprise an acidic polymer, including an ionomeric polymer. The ionomeric polymer can be a carboxylate ionomer. The acidic or ionomeric polymer can comprise an addition polymer (i.e., an acidic addition polymer) or a condensation polymer. Addition polymers include polymers prepared from reactants comprising at least one ethylenically unsaturated monomer. Ethylenically unsaturated monomers include olefin monomers such as ethylene, propylene, 1-butylene, 1-hexene, 1-octene, and 1-decene. Ethylenically unsaturated monomers also include acidic or acid-precursor monomers such as acrylic acid, itacontic acid, maleic acid, itaconic anhydride, and maleic anhydride. In some embodiments, acidic polymers are prepared from reactants comprising at least one olefin monomer and at least one acidic or acid-precursor monomer (e.g., a polymer prepared from reactants comprising ethylene and acrylic acid). Ionomeric polymers can be prepared by neutralizing the acid groups of acidic polymers (e.g., by neutralizing with a base such as sodium hydroxide). In some embodiments, the composition is free of acidic polymers.

The composition can be flexible. As used herein, the term “flexible” means that the composition can be deformed (e.g., bent, compressed, or stretched) without breaking at temperatures greater than room temperature. The composition can be sufficiently flexible or deformable such that it is capable of being inserted into a dental cavity, e.g., into a root canal. In some embodiments, a sample of the composition can be stretched to at least 100% of its length without breaking In some embodiments, the composition is flexible at the normal temperature of the human body (i.e., approximately 37° C.). The composition can be flexible at temperatures of up to 40° C., up to 50° C., up to 60° C., up to 70° C., or up to 80° C.

In some embodiments, the composition can have a melting point of no greater than 80° C. In this context, the term “melting point” refers to a temperature at which the composition becomes liquid or liquid-like (i.e., it can flow, e.g., into a root canal, under the force of gravity). The composition can have melting point of no greater than 60° C., no greater than 50° C., no greater than 40° C., no greater than 37° C., or no greater than 35° C. The composition can have a melting point of at least 35° C., at least 37° C., at least 40° C., at least 50° C., at least 60° C., at least 70° C., or at least 80° C.

The composition can be radiopaque, i.e., it can absorb as much X-ray radiation as an equivalent thickness of aluminum. In some embodiments, the composition is more radiopaque than tooth enamel. In some embodiments, the composition is more radiopaque than dentin. A cross-section of the composition can have radiopacity less than, equal to, or greater than the radiopacity of an equivalent cross-section of aluminum. The radiopacity of the composition can be measured as described in, for example, ISO 4049 §7.14 (2000).

The composition can be prepared by combining a hyperbranched compound, a polymer prepared from reactants comprising at least one (meth)acrylate monomer, and any additional component (such as a filler), heating the mixture with stirring, and allowing the mixture to cool. The mixture can be heated to at least any temperature sufficient to provide a mixture with sufficient viscosity to allow mixing by any conventional mixing method (e.g., hand mixing or mechanical mixing). The mixture can be formed into a useful shape, for example by extruding or by molding, before it is allowed to cool.

A method is provided for restoring a dental cavity, comprising providing a composition comprising a hyperbranched compound and inserting the composition into the dental cavity. The dental cavity can be a root canal. In some embodiments, the composition further comprises a polymer prepared from at least one (meth)acrylate monomer and at least one ethylenically unsaturated monomer having a polar group or a siloxane group. The (meth)acrylate monomer can comprise an alkyl, aryl, or aralkyl(meth)acrylate monomer. The composition can further comprise a filler. The filler can be a radiopaque filler.

The method can comprise inserting the composition into the dental cavity. The dental cavity, e.g., a root canal, can be shaped with hand tools or rotary tools such as files before the composition is inserted into the cavity. In some embodiments, the dental cavity is not shaped before the composition is inserted. The composition can adapt to the contours of the dental cavity. In some embodiments, the composition fills the dental cavity. The method can further comprise compacting the composition in the dental cavity. When the dental cavity is a root canal, the composition can be compacted toward the apex of the canal and can provide an apical seal. In some embodiments, the composition can be injected, for example through a hollow needle or a canula, into a root canal.

In some embodiments, the method comprises heating the composition, e.g., to soften it before inserting it into a dental cavity. The composition can be heated to a temperature greater than room temperature (i.e. greater than about 20° C.). The composition can be heated to at least 20° C., at least 30° C., at least 40° C., at least 50° C., or at least 60° C. to soften it before inserting it into a dental cavity. The composition can be heated to a temperature of no greater than 80° C., no greater than 70° C., no greater than 60° C., no greater than 50° C., or no greater than 40° C. to soften it before inserting it into a dental cavity.

In some embodiments, the composition is heated to a temperature equal to or greater than its melting point before it is inserted into a dental cavity. In these embodiments, the dental cavity can be filled by allowing the composition to flow into the dental cavity.

The composition can flow or can be compacted to conform to the contours of the dental cavity, e.g., the root canal. Surprisingly, the composition can conform to the contours of the dental cavity and provide a seal along the contours of the dental cavity. In some embodiments, a dental cavity can be filled with the composition without the use of an additional sealing agent such as zinc oxide eugenol sealing agents.

An article is provided, comprising a hyperbranched compound. In some embodiments, the article further comprises a polymer prepared from reactants comprising at least one (meth)acrylate monomer. The article can have any shape or aspect ratio, including a shape or an aspect ratio of a root canal. In this context, the term “aspect ratio” means the ratio of the length of the article to the width of the article. In the case of an article having a tapered or conical shape, the width is the widest width of the article. The article can have an aspect ratio of at least 1:1, at least 2:1, at least 3:1, at least 4:1, at least 5:1, at least 10:1, at least 20:1, at least 30:1, at least 40:1, or at least 50:1. The article can have an aspect ratio no greater than 80:1, no greater than 70:1, no greater than 60:1, no greater than 50:1, no greater than 40:1, no greater than 30:1, no greater than 20:1, no greater than 10:1, no greater than 5:1, no greater than 4:1, no greater than 3:1, or no greater than 2:1. In some embodiments, the article has a shape of a cylinder or cone. At least one cylinder or cone can be inserted into a dental cavity, e.g., a root canal. At least one cylinder or cone can fill the dental cavity. The cylinder or cone can have a unitary construction. Alternatively, the cylinder or cone can comprise a flexible or rigid core or carrier that is at least partially covered with a composition comprising a hyperbranched compound.

The article (in the shape of a cylinder or cone) can be inserted into a dental cavity (e.g., a root canal) in one piece. In some embodiments, the article can be inserted into a dental cavity in more than one piece. The article can be heated, for example by using a heated wire, after it is inserted into a dental cavity.

The article can be removed from a dental cavity. The article can comprise a composition having sufficient mechanical strength so that the article can be removed from a dental cavity in one piece (i.e., without breaking) In some embodiments, an article can be removed from a dental cavity in more than one piece. The article can be heated to a temperature at or above the melting point of the composition, and can then be removed from a dental cavity using, for example, suction via a canula. In some embodiments, the article is broken into pieces or ground into particles or a powder (e.g., using a rotary or hand tool) before it is removed from a dental cavity.

### Examples

Unless otherwise noted, reagents and solvents were or can be obtained from Sigma-Aldrich Co., St. Louis, Mo.

“BH20” refers to a dendritic polyol calculated as having hydroxyl functionality of approximately 16 and weight average molecular weight of 1750, available under the trade designation “BOLTORN H20” from Perstorp Polyols, Inc., Toledo, Ohio.

“BH40” refers to a dendritic polyol calculated as having hydroxyl functionality of approximately 64 and weight average molecular weight of 7300, available under the trade designation “BOLTORN H40” from Perstorp Polyols, Inc., Toledo, Ohio.

“VAZO 52” refers to 2,2′-azobis(2,4-dimethylvaleronitrile), available under the trade designation VAZO 52 from E.I. du Pont de Nemours and Company, Wilmington, Del.

“IBMA” refers to isobornyl methacylate.

“IBA” refers to isobornyl acrylate.

“PDMS-MA” refers to methacryloxypropyl-terminated poly(dimethylsiloxane) having a weight average molecular weight of 1500-2500, which can be obtained from Gelest, Inc., Morrisville, Pa.

“LMA” refers to lauryl methacrylate.

“MOEA” refers to 2-methoxyethyl acrylate, obtained from Polysciences, Inc., Warrington, Pa.

“PEG-MA” refers to poly(ethylene glycol) 1000 methacrylate.

“ODA” refers to octadecyl acrylate.

“NVP” refers to N-vinyl-2-pyrrolidinone.

“PEO-LE” refers to a poly(ethylene oxide)lauryl ether obtained under the trade designation BRIJ 35” from Sigma-Aldrich Co., St. Louis, Mo.

“GP-496” refers to an epoxy-functional silicone copolymer available from Genesee Polymers Corp., Burton, Mich.

“J120” refers to an oxidized poly(ethylene) wax obtained as an aqueous emulsion under the trade designation JONCRYL 120 from BASF Corp., Florham Park, N.J. The was was precipitated by adding the emulsion to ethanol, filtering the precipitate, washing the precipitate with water, and drying the precipitate in air at room temperature. The dry solid was then ground into a fine powder.

“AC285” refers to a low molecular weight ionomer obtained under the trade designation “ACLYN 285” from Honeywell International, Inc., Morristown, N.J.

“AC5180” refers to poly(ethylene-co-acrylic acid), obtained under the trade designation A-C 5180 from Honeywell International, Inc., Morristown, N.J.

“FILLER A” refers to nanoparticulate zirconia obtained from Sigma-Aldrich Co., St. Louis, Mo.

“FILLER B” refers to barium ziconate obtained from Sigma-Aldrich Co., St. Louis, Mo.

“FILLER C” refers to nanoparticulate barium strontium titanium oxide obtained from Sigma-Aldrich Co., St. Louis, Mo.

“FILLER D” refers to zirconium (IV) oxide-yttria stabilized nanopowder, obtained from Sigma-Aldrich Co., St. Louis, Mo.

### Preparative Example 1

### Preparation of Stearic Acid Ester of a Polyester Polyol

A hyperbranched polyester polyol (BH20; 50 g) was combined with toluene (approximately 150 mL) and p-toluene sulfonic acid (0.5 g) in a two neck round bottom flask fitted with a mechanical stirrer, a reflux condenser, and a Dean Stark trap. To the stirring mixture there was added stearic acid (117.04 g). The mixture was heated to reflux. Heating and stirring were continued until no additional water was collected in the trap. The mixture was then allowed to cool, and volatile components were removed using a rotary evaporator. The remaining reaction product was dried in a vacuum oven overnight at 60° C. to 70° C. to afford the product.

### Preparative Example 2

### Preparation of Stearic Acid Ester of a Polyester Polyol

Preparative Example 2 was carried out essentially as described in Preparative Example 1 except that BH40 was used in place of BH20, and 112.23 g of stearic acid was used.

### Preparative Example 3

### Preparation of Stearic Acid Ester of a Polyester Polyol

Preparative Example 3 was carried out essentially as described in Preparative Example 1 except that a mixture of stearic acid (58.52 g) and caprylic acid (29.66 g) was used in place of the stearic acid of Preparative Example 1.

### Preparative Example 4

### Preparation of Stearic Acid Ester of a Polyester Polyol

Preparative Example 2 was carried out essentially as described in Preparative Example 1 except that BH40 was used in place of BH20, and a mixture of stearic acid (56.12 g) and caprylic acid (28.44 g) was used in place of the stearic acid of Preparative Example 1.

### Preparative Examples 5-21

### Preparation of (Meth)acrylate Polymers

For each of Preparative Examples 5-21, a total of 10 grams of monomers were combined, in the weight ratios given in Table 1, with VAZO 52 (0.15 g) in screw cap vials. The vials were placed in a water bath at 50° C. After 8 hours, each vial was removed from the water bath and was allowed to cool to room temperature to afford the product.

TABLE 1
(Meth)acrylate Polymers of Preparative Examples 5-21.

| Preparative Example | IBA | PDMS-MA | LMA | MOEA | PEG-MA | IBMA | ODA | NVP |
|---|---|---|---|---|---|---|---|---|
| 5 | 6 | 1 | | | | | 3 | 1 |
| 6 | 1 | 1 | 6 | | 1 | | | |
| 7 | | 2 | 4 | | 3 | | | |
| 8 | | 2 | 4 | 2 | 2 | | | |
| 9 | 2 | 2 | 2 | 2 | 2 | | | |
| 10 | | | 2 | | | 2 | 2 | 2 |
| 11 | 2 | | | 2 | | | 2 | 2 |
| 12 | 3 | | 3 | 2 | | | | 2 |
| 13 | 1 | | 2 | 1 | | 2 | 1 | 1 |
| 14 | 1 | 1 | 2 | 1 | | 2 | 1 | |
| 15 | | | 6 | | | 1 | 1 | |
| 16 | 4 | | | | | | 4 | 1 |
| 17 | 2 | | | | | | | 1 |
| 18 | | | | 1 | | | 2 | 4 |
| 19 | | | | 1 | | 3 | 3 | 1 |
| 20 | 5 | | | | | | 4 | 1 |
| 21 | 6 | | | | | | 3 | 1 |

### Examples 1-14

To prepare the compositions of Examples 1-14, each of the components were combined in a test tube and the test tube was placed in a block on a thermostatically controlled hot plate (temperature set to 100° C. to 150° C.) for approximately 30 minutes. The softened mixture was then immediately poured into the barrel of a glass syringe. The syringe plunger was then inserted into the barrel and the softened mixture was expelled through the tip of the syringe into cold (approximately 0° C.) 95% ethanol in an aluminum dish. The expelled mixture was then cut into pieces (approximately 5 cm to approximately 10 cm in length) and the pieces were allowed to dry at room temperature. The components and amounts of each of the compositions of Examples 1-14 are given in Table 2. In Table 2, “PE1” refers to the polyester polyol product of Preparative Example 1, “PE2” refers to the polyester polyol product of Preparative Example 2, and “PE19” refers to the polymer product of Preparative Example 19. In Table 2, “ - - - ” means that the component was not present in the composition.

TABLE 2
Compositions of Examples 1-14.

| EXAMPLE | PE1 | PE2 | PE19 | AC285 | J120 | FILLER |
|---|---|---|---|---|---|---|
| 1 | 2 g | — | 1 g | 0.25 g | — | FILLER B (1.5 g) |
| 2 | — | 2 g | 0.5 g | 1 g | — | FILLER C (1.5 g) |
| 3 | 2 g | — | 0.5 g | 1 g | — | FILLER C (1.5 g) |
| 4 | — | 2 g | 0.5 g | — | 1 g | FILLER B (1.5 g) |
| 5 | — | 2 g | 0.5 g | — | 1 g | FILLER C (1.5 g) |
| 6 | — | 2.5 g | 1 g | — | — | FILLER C (1.5 g) |
| 7 | 1 g | 2 g | 0.5 g | — | — | FILLER C (1.5 g) |
| 8 | — | 2 g | 1.5 g | — | — | FILLER C (1.5 g) |
| 9 | 1 g | 1 g | 0.5 | — | 0.5 g | FILLER C (2 g) |
| 10 | — | 0.5 g | 1 g | — | 2 g | FILLER C (1.5 g) |
| 11 | 0.25 g | 0.25 g | 1 g | — | 2 g | FILLER C (1.5 g) |
| 12 | 0.25 g | 0.25 g | 1 g | — | 1.5 g | FILLER C (2 g) |
| 13 | 0.25 g | 0.25 g | 1 g | — | 1.5 g | FILLER D (2 g) |
| 14 | 1 g | 1 g | 0.5 g | — | 0.5 g | FILLER D (2 g) |

### Examples 15-18

The compositions of Examples 15-18 were prepared using the procedure essentially as described in Examples 1-14. The components and amounts of each of the compositions of Examples 15-18 are given in Table 3. In Table 3, “PE2” refers to the polyester polyol product of Preparative Example 2, and “PE19” refers to the polymer product of Preparative Example 19. In Table 3, “ - - - ” means that the component was not present in the composition.

TABLE 3
Compositions of Examples 15-19.

| | | | | PEO- | | |
|---|---|---|---|---|---|---|
| EXAMPLE | PE2 | PE19 | GP-496 | LE | J120 | FILLER |
| 15 | 2 g | 0.5 g | — | 1 g | | FILLER C (1.5 g) |
| 16 | 2.5 g | 0.5 g | 1 g | — | | FILLER C (1.5 g) |
| 17 | 1.5 g | 0.5 g | — | 0.5 g | 0.5 g | FILLER C (2 g) |
| 18 | 1.5 g | 0.5 g | — | 0.5 g | 0.5 g | FILLER D (2 g) |

### Examples 19-22

The compositions of Examples 19-22 were prepared using the procedure essentially as described in Examples 1-14. The components and amounts of each of the compositions of Examples 19-22 are given in Table 4. In Table 4, “PE3” refers to the polyester polyol product of Preparative Example 3, “PE4” refers to the polyester polyol product of Preparative Example 4, and “PE19” refers to the polymer product of Preparative Example 19. In Table 4, “ - - - ” means that the component was not present in the composition.

TABLE 4
Compositions of Examples 19-22.

| EXAMPLE | PE3 | PE4 | PE19 | J120 | FILLER |
|---|---|---|---|---|---|
| 19 | 0.5 g | — | 1 g | 1.5 g | FILLER C (2 g) |
| 20 | — | 0.5 g | 1 g | 1.5 g | FILLER C (2 g) |
| 21 | 1 g | — | 1 g | 1.5 g | FILLER C (2 g) |
| 22 | — | 1 g | 1 g | 1.5 g | FILLER C (2 g) |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A composition comprising:
a) a hyperbranched compound; and
b) a polymer prepared from reactants comprising at least one (meth)acrylate monomer.

2. The composition of claim 1 wherein the polymer is prepared from reactants further comprising at least one ethylenically unsaturated monomer having a polar group or a siloxane group.

3. The composition of claim 1 wherein the composition is substantially free of a crosslinking agent.

4. The composition of claim 1 wherein the composition is substantially free of crosslinks.

5. The composition of claim 1 wherein the composition is radiopaque.

6. The composition of claim 1 wherein the composition is substantially free of ethylenically unsaturated groups.

7. The composition of claim 1 wherein the polymer has a T_{g} no greater than 60° C.

8. The composition of claim 1 wherein the (meth)acrylate monomer comprises an alkyl(meth)acrylate monomer wherein the alkyl group comprises at least four carbon atoms.

9. The composition of claim 1 wherein the hyperbranched compound comprises at least one terminal ether, ester, amide, urea, or urethane group.

10. The composition of claim 1 further comprising a filler.

11. The composition of claim 10 wherein the filler is radiopaque.

12. The composition of claim 11 comprising at least 10 weight percent radiopaque filler.

13. The composition of claim 10 wherein the filler comprises a filler having a primary particle size no greater than 100 nanometers.

14. The composition of claim 1 wherein the hyperbranched compound comprises a hyperbranched polyester.

15. The composition of claim 1 further comprising an acidic addition polymer.

16. The composition of claim 15 wherein the acidic addition polymer is prepared from reactants comprising an olefin monomer.

17. The composition of claim 16 wherein the acidic addition polymer is further prepared from at least one acidic monomer or acid-precursor monomer.

18. The composition of claim 15 wherein the acidic addition polymer is a carboxylate ionomer.

19. The composition of claim 18 wherein the carboxylate ionomer is prepared from reactants comprising an olefin monomer.

20. A composition comprising:
a) a hyperbranched polyester compound having a plurality of terminal alkyl ester groups; and
b) a polymer prepared from reactants comprising:
i) at least one alkyl (meth)acrylate monomer; and
ii) at least one ethylenically unsaturated monomer having a polar group or a siloxane group.

21. The composition of claim 20 wherein each alkyl ester group independently comprises an alkyl group having at least eight carbon atoms.

22. The composition of claim 20 wherein the alkyl(meth)acrylate monomer comprises at least one of isobornyl acrylate, isobornyl methacrylate, octadecyl acrylate, lauryl methacrylate, or combinations thereof.

23. A method of restoring a dental cavity comprising:
a) providing a composition comprising a hyperbranched compound, and
b) inserting the composition into the dental cavity.

24. The method of claim 23 further comprising heating the composition.

25. An article for filling a root canal comprising a hyperbranched compound, wherein the article has an aspect ratio of at least 2 to 1.
